(19) United States
(12) Patent Application Publication
(10) Pub. No.: US 2021/0220434 A1
(43) Pub. Date: Jul. 22, 2021

(54) Treatment of Vascular Occlusion by Activation of Notch Signaling

(71) Applicant: The Regents of the University of California, Oakland, CA (US)

(72) Inventor: Rong Wang, San Francisco, CA (US)

(73) Assignee: The Regents of the University of California, Oakland, CA (US)

(21) Appl. No.: 17/262,460

(22) PCT Filed: Jul. 25, 2019
(86) PCT No.: PCT/US2019/043538

§ 371 Date: Jan. 22, 2021

Related U.S. Application Data
(60) Provisional application No. 62/703,872, filed on Jul. 26, 2018.

(51) Int. Cl. A61K 38/17; A61K 31/19; A61K 31/4741; A61K 31/05; A61P 9/10; A61K 35/19; A61K 35/18; A61K 47/68; A61K 31/26; A61K 31/517

(52) U.S. Cl. CPC A61K 38/177; A61K 31/19; A61K 31/4741; A61K 31/05; A61K 31/517; A61K 35/19; A61K 35/18; A61K 47/6803; A61K 31/26; A61P 9/10

(57) ABSTRACT

Provided are novel treatments for treating various conditions of insufficient blood flow or circulation. The novel inventions disclosed herein are based upon the discovery that increased Notch signaling in arterial vessels has a beneficial effect on blood flow or circulation and tissue regeneration as well as a reduction in tissue damage following arterial occlusion, constriction, or other reduction in blood flow. Increased Notch signaling in arteries promotes beneficial effects, including acute vessel dilation and/or arteriogenesis and collateral arterial growth, and improves recovery following ischemia or other reduced circulation condition. Also provided are medical devices for the delivery of Notch-activating agents to blood vessels.

### CROSS-REFERENCE TO RELATED APPLICATIONS

This application claims the benefit of priority to and is a 35 USC § 371 National Stage application of PCT/US2019/043538, entitled “Treatment of Vascular Occlusion By Activation of Notch Signaling,” filed Jul. 25, 2019, which claims the benefit of priority to U.S. Provisional Application Ser. No. 62/703,872, entitled “Improving Circulation by Notch Signaling,” filed Jul. 26, 2018; the contents of which applications are hereby incorporated by reference.

### STATEMENT REGARDING FEDERALLY SPONSORED RESEARCH OR DEVELOPMENT

This invention was made with government support under grants no. HL075033 and NS067420 awarded by The National Institutes of Health, and grant no. W81XWH-16-1-0665 awarded by the United States Army Medical Research and Materiel Command. The government has certain rights in the invention

### BACKGROUND OF THE INVENTION

Narrowing, constriction, obstruction, and/or occlusion can occur in blood vessels of the circulatory system throughout the body, including in large or small arteries, arterioles, capillaries, venules and veins. These vascular constrictions and occlusions can happen due to atherosclerosis, thrombosis, clots, embolism, vegetation, or other blockages, as well as resulting from other vascular abnormalities, decreasing blood supply to a particular tissue or organ, resulting in poor circulation, hypo-perfusion, ischemia, and/or infarct. When a constriction or occlusion happens, it can lead to conditions requiring restoration of blood flow, including arterial occlusive diseases, peripheral arterial disease, critical limb ischemia, claudication, carotid artery disease, stroke, mini-stroke, cerebrovascular disease, heart attack, coronary artery disease, and other vascular ischemic diseases. Mini-stroke, micro-infarct, or hypo-perfusion in the brain can compromise organ function, leading to or contributing to conditions such as dementia, Alzheimer's or other cognitive decline.

Constriction and/or occlusion of the arteries affects up to 35% of Americans. With increasing rates of diabetics, hypertension, and an aging population at risk of occlusive conditions, the incidence of these diseases may increase unless effective preventive and treatment strategies are developed.

In treating vascular occlusion, constriction, hypo-perfusion, insufficient blood flow, or other insufficient circulation, the objective is to restore normal blood flow and improve circulation. Currently, the treatment options for these conditions are primarily surgical procedures that aim to remove or open the blocked artery segment. For example, the current treatment for critical limb ischemia includes invasive surgical procedures such as surgical revascularization, percutaneous angioplasty, or stent placement. However, such procedures themselves intrinsically damage the blood vessels. Furthermore, many patients may not be amenable to these operations and others may require multiple and repetitive procedures. In the case of preventative care, options include lifestyle changes such as smoking cessation, increased physical activity, and weight loss, but these options have had only variable success as they require patient compliance, they act on the vasculature indirectly, and they do not change genetic, age, sex, environment, or other non-life style risk factors.

Ischemic stroke afflicts millions of people worldwide and is a leading cause of death in many nations. Stroke may be treated by thrombolytic drugs such as tissue plasminogen activator (TPA), neuroprotective drugs, and surgical intervention. Despite the therapeutic utility of these treatments, there is substantial mortality, morbidity, and expense associated with this condition and there remains a need for improved treatments for the prevention and treatment of stroke.

There are numerous other medical conditions associated with insufficient blood flow, including renal dysfunction or renal disease encompassing diminished blood flow, including as a result of atherosclerosis or diabetes, diseases of the eye involving diminished blood flow, including as a result of atherosclerosis or diabetes, and spleen infarc due to blood flow blockage.

Therefore, there is a need in the art for new preventative and treatment options to address the numerous diseases and conditions wherein reduced blood flow is implicated. unmet medical needs.

### SUMMARY OF THE INVENTION

In a first aspect, the scope of the invention encompasses novel treatments for improving blood flow or circulation to treat various conditions of insufficient blood flow or circulation. The novel inventions disclosed herein are based upon the discovery that increased Notch signaling in arterial vessels has a beneficial effect on blood flow or circulation and tissue regeneration as well as a reduction in tissue damage following arterial occlusion, constriction, or other reduction in blood flow. Increased Notch signaling in arteries promotes beneficial effects, including acute vessel dilation and/or arteriogenesis and collateral arterial growth, wherein small collateral vessels are remodeled into conduit arteries around an occlusion, which is crucial in restoring perfusion to ischemic tissue. Increased Notch signaling in arteries enhances the conductance of the vessels and reduces the resistance of vessels. Increased Notch signaling in arteries may also act by other mechanisms, leading to improved blood flow or circulation. Accordingly, increasing Notch signaling in arterial vessels can be used to treat any number of diseases and conditions encompassing vascular occlusion, constriction, insufficient blood flow, or insufficient circulation.

In one aspect, the scope of the invention encompasses a treatment of conditions characterized by loss of or reduced blood flow or circulation, for example those caused by vascular occlusion or constriction, by the administration of an agent that increase Notch signaling in arterial vessels.

In another aspect, the scope of the invention encompasses a treatment of conditions characterized by insufficient blood flow, or insufficient circulation, for example ischemia, by the administration of an agent that increase Notch signaling in arterial vessels.

In yet another aspect, the scope of the invention encompasses preventative treatments for subjects at risk of vascular occlusion or impaired circulation, by the administration of an agent that increase Notch signaling in arterial vessels.

The scope of the invention further encompasses novel medical devices for the delivery of Notch-activating agents to blood vessels.

### BRIEF DESCRIPTION OF THE DRAWINGS

FIGS. 1A and 1B. Bmx-CreERT2 mediated activation of Notch signaling through Notch1 intracellular domain (ICD) expression promotes neurological recovery, and reduces infarct volume in distal middle cerebral artery occlusion (dMCAO) mouse model. FIG. 1A: Bmx-CreERT2 mediated activation of Notch1 signaling improved neurological recovery after stroke induction by dMCAO. Neurological function was assessed by modified bederson's grading, elevated body swing test, ladder test, and adhesive test. BMX-Notch1 ICD (Bmx-CreERT2; ROSA:LNL:tTA; TRE-Notch1 ICD) mice demonstrated better neurological recovery after dMCAO surgery, compared with control mice. Triangles: BMX-Notch1 ICD, Circles: Control. Control, n=12, BMX-Notch1 ICD; n=13. *P<0.05, **P<0.01, ***P<0.001. FIG. 1B: Quantification of infarct volume showed significantly less (p<0.01) infarct volume in BMX-Notch1 ICD mice, compared with control mice. Each n=6.

FIGS. 2A and 2B. Bmx-CreERT2 activation of Notch signaling through Notch4* expression promotes neurological recovery, and reduces infarct volume in distal middle cerebral artery occlusion (dMCAO) mouse model. FIG. 2A: Activation of Notch4 improved neurological recovery after dMCAO. Neurological functions were assessed by modified bederson's grading, elevated body swing test, ladder test, and adhesive test. Triangles: BMX-Notch4*, Circles: Control. FIG. 2B: Quantification of infarct volume showed significantly less (p<0.01) infarct volume in BMX-Notch4* mice, compared with control mice. Each n=9.

FIGS. 3A and 3B. Post-strokeNotch signaling activation, through Notch1 ICD expression, initiated after ischemic insult, promoted neurological recovery and reduction of infarct volume. FIG. 3A: Bmx-CreERT2 activation of Notch1 after ischemic injury improved neurological recovery. Neurological functions were assessed by modified bederson's grading, elevated body swing test, ladder test, and adhesive test after dMCAO. Triangles: BMX-Notch1 ICD, Circles: Control. FIG. 3B: Quantification of infarct volume determined by HE staining showed significantly less infarct volume in BMX-Notch1 ICD mice, compared with control mice. Control, n=7, BMX-Notch1 ICD; n=9.

FIGS. 4A and 4B. Post stroke Notch signaling activation, through Notch4* expression, initiated after ischemic insult, promoted neurological recovery and reduction of infarct volume. FIG. 4A: Arterial activation of Notch4 after ischemic injury improved neurological recovery. Neurological functions were assessed by modified bederson's grading, elevated body swing test, ladder test, and adhesive test after dMCAO. Triangles: BMX-Notch4*, Circles: Control. FIG. 4B: Quantification of infarct volume determined by HE staining showed significantly less infarct volume in BMX-Notch4* mice, compared with control mice. Control, n=8, BMX-Notch4*; n=7.

FIGS. 5A and 5B. Arterial expression of Notch1 ICD promoted growth of collaterals, and promoted the restoration of cerebral blood flow. FIG. 5A depicts the experimental protocol for initiation of Notch1 ICD expression 14 days prior to dMCAO, and turning off of Notch1 ICD 14 days post-dMCAO. FIG. 5B: Collateral artery diameter, velocity, and flux during the experimental time course, the vertical line indicates the time at which Notch1 ICD expression was turned off. Triangles: BMX-Notch1 ICD, Circles: Control.

FIGS. 6A, 6B, and 6C. Treatment with Notch activating ligand DLL4 improves growth of collaterals and preserves cerebral blood flow following dMCAO, as well as improving neurological function. FIG. 6A depicts the time course of the experiment, with recombinant DLL4 administered one day prior to and for seven days following dMCAO stroke induction. FIG. 6B: Collateral artery diameter, velocity, and flux, and the area between the vertical lines denotes the period of DLL4 administration. Triangles: rDLL4, Circles: Control. Control, n=5, Treated; n=5. *P<0.05, **P<0.01. FIG. 6C depicts ladder test results for mice are subject to behavior test at 15 to 17 days after experimental stroke, the scores being the average of the two measurements.

FIGS. 7A, 7B, and 7C. Improved recovery in limb ischemia mouse model by expression of Notch4*. Notch 4* induction commenced 14 days prior to EFAO. FIG. 7A, Blood flow was significantly superior in BMX-Notch4* mice over the 5 weeks following EFAO. Foot perfusion is expressed as a ratio of the left (ischemic) to right (control) foot, measured by laser doppler perfusion imaging (LDPI). Triangles: BMX-Notch 4*, Circles: Control. Data are mean±SEM; **p<0.01, ***p<0.001. FIG. 7B: comparison of collateral artery diameter, 21 days following EFAO, in left and right legs for control and Notch4*-expressing mice, BMX-Notch4*. FIG. 7C, ratio in the change of collateral artery diameter after EFAO. For each, n=8, *P<0.05

FIG. 8. FIG. 8 depicts quantification of muscle necrosis at day-7 after EFAO, following two weeks of Notch4* expression, showing statistically significant reduction of necrosis in Notch4 expressing mutants.

FIG. 9. Durable improvements in foot perfusion following EFAO in mice expressing Notch4* for 14 days. Notch4 expression was initiated in mutant mice 14 days before EFAO in the left hindlimb. Foot perfusion was measured in treated left foot and untreated right foot pre- and post-EFAO. Ratio of perfusion values in treated and untreated foot was tracked over time. Notch4* expression was turned off at day 14 post EFAO. Squares: BMX-Notch4* mice, Circles: control.

FIG. 10. Significant improvements in foot perfusion following EFAO were observed in Notch4*-expressing mice. Notch4* expression was induced, immediately followed by EFAO induction in left hindlimb, by tamoxifen administration and one day following EAFO (arrows). Foot perfusion was measured in operated left side foot and unoperated right side foot pre- and post-EFAO. FIG. 10 depicts the ratio of left and right perfusion values in treated and untreated foot over time. Squares: BMX-Notch4*, Circles: control.

### DETAILED DESCRIPTION OF THE INVENTION

The scope of the invention encompasses the treatment of a vascular condition by the administration of an agent that activates Notch signaling in blood vessels. In one embodiment, the scope of the invention encompasses an agent that activates Notch signaling in blood vessels for use in the treatment of a vascular condition. In a related embodiment, the scope of the invention encompasses a method of treating a vascular condition in a subject in need of treatment therefor by the administration of a pharmaceutically effective amount of an agent that activates Notch signaling in blood vessels. In a related embodiment, the scope of the invention encompasses a method of using an agent that activates Notch signaling in blood vessels in the manufacture of a medicament for the treatment of a vascular occlusion or constriction condition. The various elements of the invention are described next.

Treatment.

The various embodiments of the invention are directed to the treatment of vascular conditions, as defined below. “Treatment,” as used herein, will encompass any preventative or therapeutic treatment. In a first aspect, “treatment” will encompass inducing a therapeutic effect with respect to one or more vascular conditions, including, for example: inhibiting a process that underlies a vascular condition; ameliorating the symptoms of a vascular condition; slowing the progression of a vascular condition; reducing the severity of a vascular condition; and curing a vascular condition.

In some implementations, “treatment,” as used herein, will encompass preventative treatment, for example, a treatment which achieves one or more of the following: prevent the onset of a vascular condition; delay, slow or halt the progression of a vascular condition; improve vascular circulation or health; reduce the risk of a vascular condition; slow or halt the progression of an ischemic condition; and, increase or restore Notch signaling in blood vessels.

In some implementations, “treatment,” as used herein, means achieving one or more physiological, physical, functional, therapeutic, or performance outcomes. For example, treatment may encompass: increasing Notch signaling in one or more blood vessels; improving blood flow or circulation through one or more blood vessels; promoting arteriogenesis; dilating one or more blood vessels; enhancing the conductance of one or more blood vessels; reducing the resistance of one or more blood vessels; and improving vascular tone of one or more blood vessels.

The treatments of the invention may achieve local effects, for example, improving blood flow at an ischemic site or in an organ, or may achieve systemic effects, for example, improving circulation generally throughout the body.

Vascular Conditions.

The various implementations of the invention are directed to treating vascular conditions. A “vascular condition,” as used herein, may comprise any disease, condition, or pathology, including both acute and chronic conditions, wherein blood flow is reduced, impeded, or blocked in one or more blood vessels. A vascular condition may comprise a condition that constitutes a reduction, impediment, or blockage of blood flow. A vascular condition may further comprise a condition resulting in, or resulting from reduced, impeded, or blocked blood flow.

In one aspect, the vascular condition is ischemia. In one implementation, the ischemia is cardiac ischemia, also known as coronary artery disease, and also known as ischemic heart disease. Cardiac ischemia encompasses, stable angina, unstable angina, acute coronary syndrome, angina pectoris, myocardial infarction, and ischemia resulting from atherosclerosis.

In one aspect, the vascular condition is ischemia of the brain. In one implementation, the brain ischemia is acute ischemic stroke. In one embodiment, the brain ischemia is transient ischemic attack, or mini-stroke. In one embodiment, the brain ischemia is or micro-infarct. In one embodiment, the brain ischemia is vascular dementia. In one embodiment, the vascular condition is cerebrovascular disease. In one embodiment, the vascular condition is a condition encompassing reduced circulation in the brain, including, for example, dementia, Alzheimer's, or other cognitive decline.

In one embodiment, the vascular condition is ischemia of the limbs. In one embodiment, the limb ischemia is critical limb ischemia.

In one embodiment, the vascular condition is an ischemia of the bowel.

In one embodiment, the vascular condition is carotid artery disease.

In one embodiment, the vascular condition is peripheral arterial disease. In one embodiment, the peripheral artery disease is critical limb ischemia. In one embodiment, the peripheral artery disease is claudication.

In one embodiment, the vascular condition is renal dysfunction or renal disease encompassing diminished blood flow, including as a result of atherosclerosis or diabetes.

In one embodiment, the vascular condition is a condition of the eye involving diminished blood flow, including as a result of atherosclerosis or diabetes.

In one embodiment, the vascular condition involves diminished blood flow in the spleen, including spleen infarc due to blood flow blockage.

In one embodiment, the vascular condition is mesenteric artery occlusion or intestinal infraction.

In one embodiment, the vascular condition is moyamoya disease.

In one embodiment, the vascular condition is Cerebral autosomal dominant arteriopathy with subcortical infarcts and leukoencephalopathy, sometimes referred to CADASIL

in one embodiment, the vascular condition is Alagille syndrome or Alagille-Watson syndrome, including inherited or spontaneous forms thereof for example manifesting in the liver, heart, kidney, and other systems of the body. ALGS is caused by loss of function mutations in either JAG1 or NOTCH2.

In one embodiment, the vascular condition is small vessel disease, small vessel infarction, or white matter disease.

In one embodiment, the vascular condition is the need for establishing or improving blood flow following receipt of grafted tissue, such as kidney, liver, lung, heart, cellular grafts.

In one embodiment, the vascular condition is the need for establish or improving blood flow in areas of regenerating tissue.

The methods of the invention are especially amenable to the treatment of vascular occlusion or constriction in the arteries. In one embodiment, the vascular condition is a condition encompassing reduced circulation in the small arteries or arterioles, wherein it can also cause poor circulation and compromise organ function. In other embodiments, the vascular condition manifests in the veins, capillaries, or in grafted blood vessels.

In one embodiment, the vascular condition is an arterial occlusive disease in any part of the body. In one embodiment, the vascular condition is hypoperfusion. In one embodiment, the vascular condition is atherosclerosis. In one embodiment, the vascular condition is thrombosis. In one embodiment, the vascular condition is the formation or persistence of clots. In one embodiment, the vascular condition is embolism. In one embodiment, the vascular condition is pulmonary embolism. In one embodiment, the vascular condition is vegetative or other blockage of the blood vessels.

Subjects.

The methods of the invention are applied in the treatment of a subject. The subject will be a subject in need of treatment of a vascular condition, for example, a subject suffering from a vascular condition or at risk of a vascular condition. The subject may be any animal, for example, a human subject, a non-human primate, a mouse, rat, other rodent, dog, cat, cow, pig, horse, or any other animal species. In one embodiment, the subject is a human patient. In one embodiment, the subject is a veterinary subject. In one embodiment, the subject is a test animal.

In one embodiment, the subject is a subject at risk of a vascular condition. In one embodiment, the subject at risk of a vascular condition is an aged subject. For example, in the case of human subjects, an aged subject may be at least 40 years old, at least 45 years old, at least 50 years old, at least 55 years old, at least 60 years old, at least 65 years old, at least 70 years old, at 80 years old, or older. In other embodiments, the subject at risk of a vascular condition is a smoker or ex-smoker. In other embodiments, the subject at risk of a vascular condition is a subject with a medical or family history of vascular disease. In other embodiments, the subject at risk of a vascular condition is overweight or obese, for example, having a BMI greater than 25, or greater than 30. In one embodiment, the subject at risk is a subject having diabetes. In one embodiment, the subject at risk is a subject having hypertension.

Notch Signaling Activation.

The scope of the invention encompasses the use of agents which are activators of Notch signaling. The Notch receptor is a single-pass transmembrane receptor protein. Mammalian Notch receptors encompass Notch1, Notch2, Notch3, and Notch4, with Notch1 and Notch4 being expressed in arterial endothelial cells.

The Notch pathway mediates cell-to-cell signaling by which an array of cell fate decisions in neuronal, vascular, cardiac, immune, endocrine development, and other processes are regulated. In Notch signaling, Notch receptors on the surface of the cell interact with transmembrane ligands present on the surface of adjacent cells. Ligand binding then leads to a sequence of proteolytic cleavages of the Notch receptor releasing the Notch intracellular domain (ICD) from the membrane. The ICD translocates into the nucleus where it enters into a complex with nuclear proteins, CSL protein CBF-1/RBPJ in mammals, to regulate transcription of various target genes. Through RBPJ, Notch activates canonical signaling. Notch also activates non-canonical signaling through other downstream effectors.

As used herein, “activation of Notch” or “Notch activation,” referred to herein as “activation of Notch signaling,” encompasses the induction, increase, or upregulation of any Notch signaling activity or effect, canonical or non-canonical, as known in the art. The activation of Notch signaling referred to herein includes enhancement of existing endogenous Notch activity, restoration of normal endogenous Notch activity, and enhancement and addition of Notch activity in arterial vessels. Notch signaling may encompass, for example: cleavage of Notch to release the ICD, translocation of the ICD to the nucleus, increasing TNF-α ADAM metalloprotease converting enzyme (TACE) activity; increasing ubiquitination of the cleaved Notch extracellular domain by Mib, increasing γ-secretase mediated release of the Notch intracellular domain, enhancing CBF1/Su(H)/Lag-1 transcription factor complex formation or activity; and/or modulation of Notch downstream genes consistent with endogenous Notch activity (either by the ICD or by agents that bypass and mimic one or more effects of the ICD). Downstream genes include Deltex-1, Deltex-2, Deltex-3, Suppressor of Deltex (SuDx), Numb and isoforms thereof, Numb associated Kinase (NAK), Notchless, Dishevelled (Dsh), emb5, Fringe genes (such as Radical, Lunatic and Manic), PON, LNX, Disabled, Numblike, Nur77, NFkB2, Mirror, Warthog, Engrailed-1 and Engrailed-2, Lip-1 and homologues thereof, the polypeptides involved in the Ras/MAPK cascade modulated by Deltex, epherin-B2, Myc, p21, and HES-family members, ephrin-B2, Eph-B4, connexin 40, FBW7, and other Notch target genes. Notch signaling activation ultimately leads to gene expression changes (up or down regulation) of downstream target genes.

Notch activation will further encompass, for example, an increase in Notch activity in target cells; an increase in expression of a Notch gene or protein, a notch ligand gene or protein, a notch positive regulator gene or protein, a Notch signaling mediator gene or protein, a Notch signaling modulator gene or protein, or Notch downstream targets in target cells; an upregulation in Notch signaling; an increase in the expression or activity of Notch-activated downstream species or effectors; the inhibition of a negative regulator of Notch signaling, or any other increase in Notch signaling in beneficial manner in target cells, regardless of mode of action.

Notch activation, as used herein, will encompass any activation of a Notch signaling pathway, including pathways mediated by Notch 1, Notch 2, Notch 3, and Notch 4, via canonical or non-canonical signaling. In a primary embodiment, as Notch 1 and Notch 4 are the isoforms primarily expressed in arterial endothelial cells, Notch activation will refer to Notch 1 and/or Notch 4 activation.

Notch activation, as used herein, will encompass Notch signaling activation in any cell type of the body. In a primary implementation, Notch signaling activation will refer to activation of Notch signaling pathways in blood vessel cells, including in the endothelial, or smooth muscle cells of blood vessels. In a primary implementation, Notch signaling activation will refer to activation of Notch signaling pathways in arterial cells, for example, arterial endothelial cells. Without being bound to any particular theory, it is believed that the activation of Notch in vascular endothelial cells is primarily responsible for the therapeutic effects described herein. However, it will be understood that the therapeutic effects of Notch activation may result from Notch activation in other cell types, for example smooth muscle cells or blood cells, or in other areas of the body, and the scope of the invention is not limited to therapeutic effects of Notch activation in arterial endothelial cells.

Notch-Activating Agent.

The scope of the invention encompasses the administration of a Notch-activating agent. The Notch-activating agent comprises any composition of matter which increases Notch signaling in cells of the body, for example, blood vessel endothelial cells, for example arterial endothelial cells. The Notch activating agent may comprise any agent having Notch-activating activity, including, for example, antibodies, small molecules, peptides and proteins, and nucleic acids.

In one implementation, the Notch-activating agent is a small molecule. Exemplary small molecule Notch agonists include N-methylhemeanthidine chloride, valproic acid, resveratrol, triacetyl resveratrol, phenethyl isothiocyanate, and Yhhu-3792, and Notch-activating chemical analogs and variants of the foregoing.

In one implementation, the Notch-activating agent is a peptide or protein. In a first implementation, the peptide or protein is a Notch ligand, i.e. a species that in vivo is expressed on the cell surface and that interacts with Notch and activates Notch signaling upon binding to a Notch extracellular domain. Notch ligands include any mammalian Notch ligand, for example, Delta-like ligands, including Delta-like ligand 1 (DLL1), Delta-like ligand 2 (DLL2) Delta-like 3 (DLL3) Delta-like ligand 4 (DLL4). Notch ligands further include the mammalian Jagged ligands Jagged-1 and Jagged-2. The Notch ligand may further comprise a non-mammalian Notch ligand or variant thereof, for example, Delta proteins, proteins of the Serrate family, (including Serrate-1 and Serrate-2) and LAG-2.

The Notch ligand may comprise variants of known Notch ligands, for example proteins having at least 70%, at least 75%, at least 80%, at least 85%, at least 90%, at least 95%, or at least 99% sequence identity to a Notch ligand while retaining or enhancing Notch activation activity. The Notch ligand variants may also comprise truncations of the wild type proteins. For example, in one embodiment, the Notch-activating ligand variant comprises the highly conserved Jagged1 delta/Serrate/Lag-2 (DSL) domain. In another embodiment, the Notch-activating ligand variant comprises amino acid residues 188-204 of human Jagged 1, which has been shown to have Notch activating activity, for example as described in Kannan et al. (2013) Notch activation inhibits AML growth and survival: a potential therapeutic approach. J Exp Med 210:321-337. Wild type DLL ligands comprise eight epidermal growth factor like (EGF-like) sequence repeats, while wild type Jagged ligands comprise twelve EGF-like repeats. Notch ligand variants of the invention may comprise variants of DLL or Jagged having altered numbers of EGF-like sequence repeats compared to wild type sequences. Furthermore, the Notch ligand variants may comprise hybrid proteins made up of subunits from two or more ligand types, for example, as described in Tveriakhina et al., The ectodomains determine ligand function in vivo and selectivity of DLL1 and DLL4 toward NOTCH1 and NOTCH2 in vitro eLife 2018; 7:e40045. For example, In one embodiment the Notch ligand variant comprises the N-terminal MNNL and DSL domains and adjacent EGF repeats 1-3 of DLL1, DLL2, DLL3, DLL4, Jagged 1, or Jagged 2. In another embodiment, the Notch ligand variant comprises a DLL4 mutant comprising an arginine to proline substitution at positions 257, as described in Liu et al., Identification of Domains for Efficient Notch Signaling Activity in Immobilized Notch Ligand Proteins, J Cell Biochem 2017 April; 118(4):785-796.

In one implementation, the peptide or protein activator of Notch is a Notch ligand mimic, including engineered variants and de novo synthetic molecules comprising amino acid sequences, peptides, and proteins that have Notch activating activity. For example, the engineered variants may comprise ligand binding domains of Notch ligands and other active domains thereof, for example, altered to have increased activity.

In one implementation, the peptide or protein activator of Notch is a Notch ICD, for example, a Notch 1, Notch 2, Notch 3, or Notch 4 ICD. The ICD may be fused or conjugated to carriers for trafficking into or across the membrane, such as lipid carriers or peptide species that facilitate transmembrane transport. For example, the ICD may be fused to a trans-activator of transcription (TAT) peptide, a penetratin, cholesterol-dependent cytolysin, or other cell penetrating peptide.

In one implementation, the Notch activating peptide or protein comprises an antibody, or antigen binding fragment thereof, wherein the antibody binds to Notch and initiates ICD cleavage and/or Notch activation. For example, in one embodiment, the Notch-activating agent comprises antibody A13 as described in Li et al., Modulation of Notch Signaling by Antibodies Specific for the Extracellular Negative Regulatory Region of NOTCH3, J Bio Chem 283: 8046-8054, 2008.

In one implementation, the Notch-activating agent comprises a lipid. Jagged and DLL ligands contain lipid binding motifs, wherein binding of certain lipids modulates Notch activation. In one embodiment, the lipid having Notch activation activity is a lipid which binds to Notch or to a Notch ligand. For example, In one embodiment, the Notch activating lipid comprises shingosine-1-phosphate (S1P). Similarly, the S1P receptor 3 protein may be used.

In one embodiment, the Notch-activating agent is a nucleic acid, for example a genetic construct which is delivered to target cells and expressed by such cells. In one embodiment, the Notch-activating agent is a nucleic acid construct that codes for a Notch-activating protein, or a downstream effector of Notch signaling. In one embodiment, the construct codes for a Notch receptor (e.g. Notch 1, Notch 2, Notch3, or Notch 4). In one embodiment, the Notch receptor is Notch4*, which, as used herein, denotes the Notch Flk1/int-3 allele is a gain-of-function Notch4 mutation mutant, as known in the art, producing a Notch 4 protein wherein the intracellular domain is constitutively cleaved following expression. In one embodiment, the construct codes for a Notch intracellular domain, for example, lacking an extracellular domain, for example, a Notch 1 or Notch 4 intracellular domain. In one embodiment, the construct codes for a Notch ligand, for example, DLL1, DLL2, DLL3, DLL4, Jagged 1, Jagged 2, or variants of a Notch ligand.

The Notch activating genetic construct may comprise an expression vector of any type, including, for example, a gene construct delivered by gene therapy technologies: viral vector (e.g. adenovirus or adeno-associated virus, lentivirus), clustered regularly interspaced short palindromic repeats-associated nuclease system (CRISPR/Cas) type constructs, CRISPRa, nanoparticle mediated gene delivery (e.g. dendrimers, lipids, chitosan gene delivery particles, etc.) or any other gene therapy constructs known in the art. The genetic construct may further comprise a constitutive promoter for high levels of Notch activator expression or an inducible promoter for controlled expression of a gene in Notch signaling pathway. The promoter may be tissue-specific promoter, for example, an arterial-specific promoter, in humans, promoters such as the fms-like tyrosine kinase-1 (FLT-1), intercellular adhesion molecule-2 (ICAM-2), and von Willebrand factor (vWF) promoters, DLL4, Notch1, Notch4, connexin 40, connexin 43, connexin 37, In murine subjects, promoters such as BMX may be used.

In one embodiment, the Notch-activating agent is an inhibitor of a negative regulator of Notch, for example an inhibitor of Ubiquitin Ligase RNF8, NUMB, SEL-10, and FBW7. The inhibitor may be a small molecule, peptide, amino acid, or other composition of matter that downregulates or inhibits a negative regulator of Notch activation and wherein inhibition of the negative regulator causes an induction or increase in Notch signaling.

In one embodiment, the Notch-activating agent is an RNA that affects Notch activation, such as a microRNA, RNAi construct, short hairpin RNA or other RNA sequence that can increase Notch signaling. For example, the RNA may comprise a micro-RNA or other RNA that inhibits expression or activity of a negative regulator of Notch signaling. The RNA construct may comprise a transient expression vector for the expression of Notch activating proteins.

Nucleic acid construct delivery to target cells, for example, endothelial cells of the artery, may be achieved by any means known in the art. For example, delivery may be achieved by viral gene vectors, electroporation, biolistic delivery systems, microinjection, ultrasound, hydrodynamic delivery, liposomal delivery, polymeric or protein-based cationic agents (e.g. polyethylene imine, polylysine), intraject systems, and DNA-delivery dendrimers. Gene delivery may be systemic (e.g. intravenous), or localized, for example, by localized injection, delivery by catheters, such as drug eluting balloon catheters, or by drug eluting implants such as stents. Liposomal delivery systems may also be used.

For example, by methods of delivering transgenes to be expressed in vascular tissues. Methods for targeted delivery to blood vessels may be adapted from methods known in the art, for example, those described in United States Patent Application Publication Number 20050053590, entitled “Endothelium-targeting nanoparticle for reversing endothelial dysfunction,” by Meininger; PCT International Patent Application Publication Number 2002042426, entitled “Carrier system for specific artery wall gene delivery,” by Yu et al.; and United States Patent Application Publication Number 20090209630, entitled “Gene delivery formulations and methods for treatment of ischemic conditions,” by Coleman et al.

In one embodiment, the Notch activator is an herbal or plant-based composition. For example, rain lily (Zephyranthos candida) extracts have the Notch-activating molecule N-methylhemeanthidine chloride, for example, as described in Ye et al., Small molecule activation of NOTCH signaling inhibits acute myeloid leukemia Scientific Reports volume 6, Article number: 26510.

Notch Activating Agent Constructs, Formulations, and Administration.

The Notch activating agents of the invention may be configured and formulated for enhanced efficacy, and may be combined with devices or other agents.

The Notch-activating agents of the invention may be administered in combination with pharmaceutically acceptable excipients, carriers, diluents, release formulations and other drug delivery vehicles, as known in the art.

The Notch activating agents of the invention may comprise targeting moieties, being compositions of matter that target a Notch-activating species to the target vascular cells. In some implementations, the methods of the invention are applied in any arterial vessel, including arteries and arterioles. In one embodiment the method of the invention comprises the administration of an agent that can increase Notch signaling selectively or preferentially in arterial cells, including arterial endothelial cells. Increasing Notch signaling selectively or preferentially in arterial cells may encompass enhanced Notch signaling that is targeted to arterial cells, is insignificant or absent in non-arterial blood vessels and other non-target tissues, or that is otherwise of greater magnitude or duration in arterial cells than in non-arterial blood vessels.

Target vascular cells may further include other vascular cells in other vessel types, such as veins, including endothelial cells, smooth muscle cells, and blood cells. For example, targeting moieties that bind to ligands presented during stress or inflammation may be used for targeting Notch-activating agents to diseased or damaged endothelial cells. Exemplary targeting moieties include, for example, leukocyte-adhesion molecules, LDLs, and derived phospholipids. Other ligands present in blood vessels, for example, arterial vessels, may be used to target Notch-activating agents to the relevant cell types.

In some embodiments, the Notch activating agent is delivered substantially alone in buffer, saline, or water without excipients. Advantageously, as described in the Examples section, Notch-activating agents delivered intravenously appear to primarily activate Notch in the arterial endothelium, absent specific targeting moieties.

In some embodiments, the Notch-activating agent is delivered by liposome. Liposomal delivery systems are amenable to the delivery of nucleic acids, peptides, and other agents. Arterial delivery of agents is described, for example, in Hwang et al., Improving Cerebral Blood Flow Through Liposomal Delivery of Angiogenic Peptides: Potential of ¹⁸F-FDG PET Imaging in Ischemic Stroke Treatment J Nucl Med. 2015; 56:1106-1111.

In some embodiments, microbubble delivery methods, as known in the art, may be utilized, including ultrasound meditated microbubble delivery, for site-specific delivery.

In some embodiments, the Notch-activating agent is delivered by nanoparticle-loaded films, as known in the art, for example, films that are wrapped around a target blood vessel.

In some embodiments, the Notch-activating agent is delivered by drug-antibody conjugate technology, wherein the Notch-activating agent is conjugated to an antibody or antigen-binding fragment thereof, such as a binding fragment developed by phage display, wherein the antibody or antigen binding fragment selectively binds ligand present in arteries or other target cell types. For example, targeting moieties may be directed to CD34, Adhesion Molecule 1, and other arterial ligands, for example, ligands found in injured blood vessels such as cross linked fibrin.

In one embodiment, the Notch-activating agent comprises a Notch-activating composition chemically conjugated to magnetizable microparticles or nanoparticles (e.g. iron oxide, Ferroferric oxide), for use in magnetic directed drug delivery methods, as known in the art.

In some implementations, the targeting moiety and the Notch-activating composition are both proteins or peptides. In such implementations, the Notch-activating agent comprises a fusion protein comprising a targeting moiety and a Notch-activating protein or peptide.

In one implementation, the Notch-activating agents of the invention are coated onto, conjugated to, or otherwise present on a device. Devices may be delivered to the target site by transcatheter delivery, as known in the art.

In one implementation, the device may be coated with a formulation of Notch-activating agent admixed with a polymeric material for timed release elution of the agent, as known in the art. Exemplary drug eluting polymers may comprise materials known in the art, such as polyurethanes, polyclones, polymethyl methacrylates, polyvinyl alcohols, and polyethylenes. In an alternative embodiment, the Notch-activating agent is conjugated directly to the surface of the device.

For example, in one embodiment, the device may comprise an implant. In one embodiment, the implant may comprise a stent. The stent may be a metal stent or polymeric stent, for example, a biodegradable or resorbable polymeric stent.

In one embodiment, the device may comprise a drug coated balloon, as known in the art. Drug coated balloons comprise a polymeric balloon deployed from a catheter and, once positions, for example by the aid of radiographic imaging, the balloon is inflated such that it contacts the vessel wall and exposes the vessel wall to an agent coated on the outside of the balloon, typically in a carrier or excipient such as polysorbate, sorbitol, iopromide, butyryl-tri-hexyl citrate (BTHC), aleuritic acid, and shelloic acid. The balloon may be left for a period of time to effect efficient transfer to arterial walls.

In some implementations, the Notch-activating agents are formulated within or on drug delivery particle compositions, such as microspheres, nanospheres, nanoparticles, vesicles, synthetic exosomes, and other drug delivery particles.

In one implementation, the Notch-activating agents are conjugated to red blood cells, platelets, or synthetic mimics of red blood cells or platelets. Red blood cell modification methods are known in the art, including, for example, as described Shi et al., Engineered red blood cells as carriers for systemic delivery of a wide array of functional probes, PNAS 2014 28:10131-10136. Likewise, functionalized platelets maybe used, for example, as reviewed by Lu et. al., Platelet for Drug Delivery 2014, Curr Op Biotech 58:81-91.

The Notch-activating agent may be co-administered in combination with any other therapeutic composition, for example: a thrombolytic drug, such as tissue plasminogen activator; a neuroprotective drug; antihypertensives; anticonvulsants; adrenergic receptor antagonists; and other agents administered in the treatment of ischemia or other vascular conditions.

Administration of Notch Activating Agents.

In the treatments of the invention, the Notch-activating agent is delivered in a pharmaceutically effective amount to one or more sites in the body for the treatment of a selected vascular condition. A pharmaceutically-effective amount is any amount sufficient to induce a measurable therapeutic effect or measurable Notch signaling activity.

The method and timing of administration will be selected based on various factors: the vascular condition at issue, the progression and status of the vascular condition, the status of the subject; the physical and pharmacological properties of the selected Notch-activating agent; and the delivery method.

For example, localized or systemic delivery of Notch-activating agents may be performed. In one implementation, systemic administration, for example, to the circulatory system is achieved. Systemic administration may be desirable in the case of preventative treatments, for example, to promote arterial resiliency in subjects at risk of a vascular condition, for example, preventative treatment of ischemia or another vascular condition in an aged subject. Systemic delivery may also be performed when the Notch-activating agent has minimal or acceptable off-target effects. In other contexts, localized delivery is preferred, for example, in the treatment of an acute injury, occlusion, or other localized condition, for example, wherein access to a constricted area by catheters or other devices is problematic.

Administration of the agent may be long term, for example, in the case of a preventative treatment to create or maintain a lowered risk of the vascular condition, or in the treatment of a chronic condition. Administration may be short term, for example, in the treatment of an acute vascular condition, such as ischemia.

In a primary implementation, given that the target cells are blood vessel cells, the administration of the Notch-activating agent will be by intravenous route. In alternative embodiments, the administration may be oral, topical, by intraperitoneal injection, or otherwise as compatible with the selected Notch-activating agent.

In a first implementation, the Notch-activating agent is applied systemically by intravenous injection. In such case, the agent is readily exposed to target endothelial cells of the circulatory system. This route of administration advantageously enables delivery to vessels deep within the body or which are otherwise not readily accessible to surgical intervention.

In alternative implementations, the Notch-activating agent is delivered locally, for example, by injection to an afflicted blood vessel. For example, the Notch-activating agent may be delivered to the sections of vessel immediately upstream and/or downstream of an afflicted area or within the afflicted area of the vessel, for example, an occluded section or infarct.

In one embodiment, the Notch-activating agent is delivered locally to an afflicted vessel by a catheter or like device introduced into the vessel. The Notch-activating agent may be flowed or dispensed from the catheter, or may be present on the catheter surface, or in a drug eluting structure, e.g., balloon, delivered to the afflicted site by the catheter.

In another embodiment, the Notch-activating agent is administered in combination with a surgical implant. For example, in one embodiment, the Notch-activating agent is administered on a stent or other implant wherein the implant is coated with a Notch-activating agent in a drug-eluting delivery vehicle or formulation.

Appropriate dosages and treatment regimens will vary and may be selected by one of skill based on patient, disease, agent, and delivery factors. Exemplary dosages of 1 nanomolar to 100 micromolar may be administered, for example, dosages of 1-1000 micrograms agent per kilogram body weight per day, for example, about 50-100 micrograms agent per kilogram body weight per day. Dosages may be administered multiple times per day, daily, every other day, or weekly, or continuously, for example, in the case of drug eluting pumps or devices. Dosages may be administered for a period of days, weeks, months, or longer.

### EXAMPLES

### Example 1. Mice with Inducible Arterial Notch Signaling

To study the effects of Notch signaling in mouse models of ischemia, constitutively active forms of Notch1 and Notch4 were used, denoted Notch1 ICD and Notch4*, respectively. Notch1 ICD comprises a truncated Notch1 receptor intracellular domain (ICD) without the transmembrane or extracellular domains, functionally replicating a cleaved Notch intracellular domain and being constitutively active upon expression. The Notch4* mutant comprises a truncated Notch4 receptor with transmembrane and intracellular domains without the extracellular domain, and it is constitutively cleaved into Notch4 ICD. Therefore, Notch4* is functionally constitutively active upon expression. Notch1 ICD or Notch4* thus increase Notch signaling when expressed in a cell.

The expression of these two mutant Notch genes were placed under the Tetracycline (Tet) Response Element (TRE), which is activated by tet transactivator (tTA), in a tetracycline (tet)-OFF gene expression system, TRE-Notch1 ICD and TRE-Notch4*. tTA is expressed following Cre activation in ROSA:LNL:tTA allele. Thus the expression of Notch1 ICD and Notch4* are under the control of the Bmx(PAC)-CreER^{T2} transgene. Bmx(PAC)-CreER^{T2} is primarily active in arterial endothelial cells, with reporter studies also showing some patchy detectable expression in brain veins and capillaries, and some patchy detectable expression in limb veins, with no substantial expression detected in limb capillaries. This construct was coupled with the tamoxifen inducible system and tetracycline (tet)-OFF gene expression system. Tamoxifen administration activates Bmx(PAC)-CreER^{T2} and leads to tTA expression. tTA is active (and thus gene expression is ON) in the absence of tetracycline. In this system, if the animals remain untreated with tetracycline or derivatives thereof, the induction of transgene expression is solely controlled by Tamoxifen administration. For gene induction, Tamoxifen was given to adult mice on two consecutive days. [Tamoxifen injection is once a day for two days, by IP injection. Injections at 14 and 13 days before dMCAO or EFAO were administered in some treatments. In other treatments, induction of Notch signaling was initiated at the time of Occlusive event (dMCAO or EFAO), being administered at Day 0 (day of Occlusion) and Day 1 after dMCAO or EFAO.

Mice carrying Bmx(PAC)-CreER^{T2}; ROSA:LNL:tTA; TRE-Notch1 ICD and are referred to herein as BMX-Notch1 ICD mice and mice carrying Bmx(PAC)-CreER^{T2}; ROSA:LNL:tTA; TRE-Notch4* are referred to herein as BMX-Notch4* mice. Control mice are mice carrying the Bmx(PAC)-CreER^{T2} and/or ROSA:LNL:tTA construct, with no Notch.

In Notch4* expressing mice, Notch4* staining studies in the limb show colocalization of Notch4* with vascular endothelial cells, but expression of Notch4* in other cell types cannot be ruled out.

Examination of membrane-targeted green fluorescent protein produced by the Rosa26-mT/mG reporter revealed Bmx(PAC)-CreER^{T2} activity present primarily in arteries, strong in the endothelial cells (ECs) of arteries, weak and sporadic in cells of veins, and absent in capillaries of the hindlimb. Analysis of multiple tissues revealed Bmx(PAC)-CreER^{T2} activity was present primarily in cells of arteries in all cases. In addition, Bmx(PAC)-CreER^{T2} activity was also present, to a lesser extent, in venous and lymphatic ECs of the ear, lymphatic ECs of the mesentery, and venous ECs of the cremaster muscle. None of the tissues displayed significant Bmx(PAC)-CreER^{T2} expression in capillary beds.

### Example 2. Artery Activation of Notch Signaling Through Notch1 Intracellular Domain (ICD) Expression Promotes Recovery in a Mouse Model of Ischemic Stroke

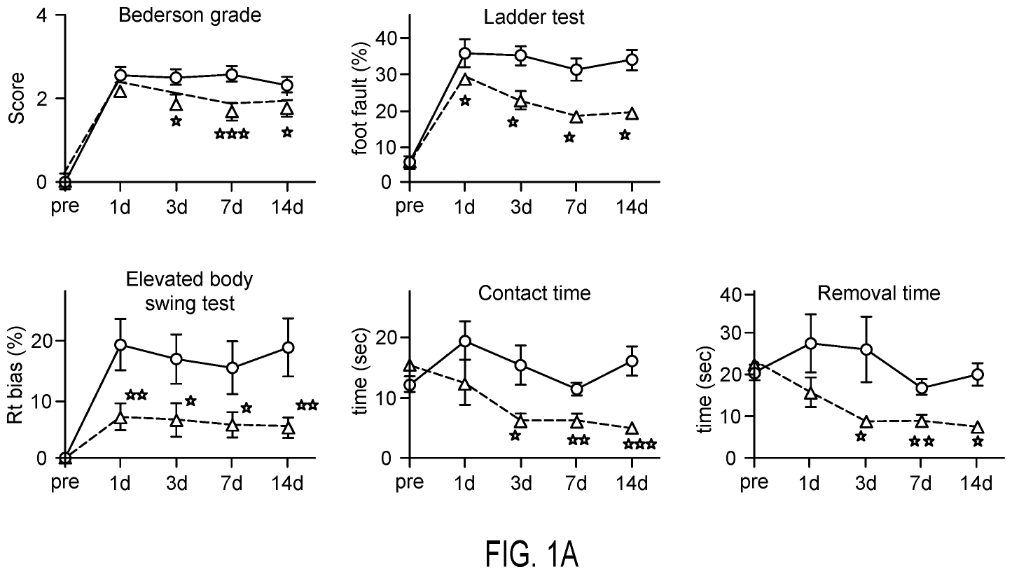

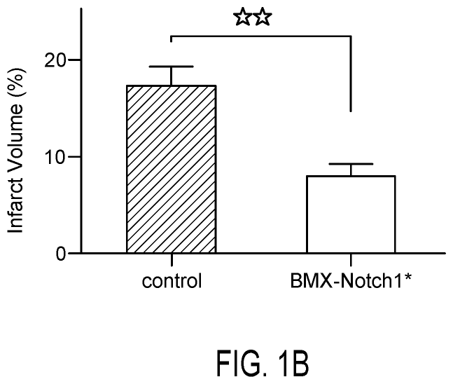

Notch1 ICD expression was induced in BMX-Notch1 ICD mice by administration of tamoxifen, 14 and 13 days prior to experimental stroke induced by dMCAO procedure as known in the art. Control and BMX-Notch1 mice were subjected to dMCAO procedure. Neurological function was tested pre- and post-dMCAO. Arterial activation of Notch1 improved neurological recovery after dMCAO. Neurological functions were assessed (FIG. 1A), including by modified bederson's grading, elevated body swing test, ladder test, and adhesive test. BMX-Notch1 ICD mice demonstrated better neurological recovery after dMCAO surgery, compared with control mice. H&E staining showed less infarct lesions in BMX-Notch1 ICD mice, compared with control mice. Quantification of infarct volume showed significantly less (p<0.01) infarct volume in BMX-Notch1 ICD mice, compared with control mice. (FIG. 1B).

Furthermore, vascular casting of [remove all pial throughout] arteries showed more prominent remodeling of collaterals (anterior cerebral artery-middle cerebral artery anastomosis) in the brains of BMX-Notch1 ICD mice compared to control mice. Although remodeling of collaterals in the ipsilateral side of dMCAO were observed in both BMX-Notch1 ICD and control mice, the extent of remodeling in vessel number, diameter and tortuosity was more prominent in BMX-Notch1 ICD mice. Enlarged images clearly showed that pial collaterals in BMX-Notch1 ICD expressing mice are more prominent and tortuous, compared to that of control mice.

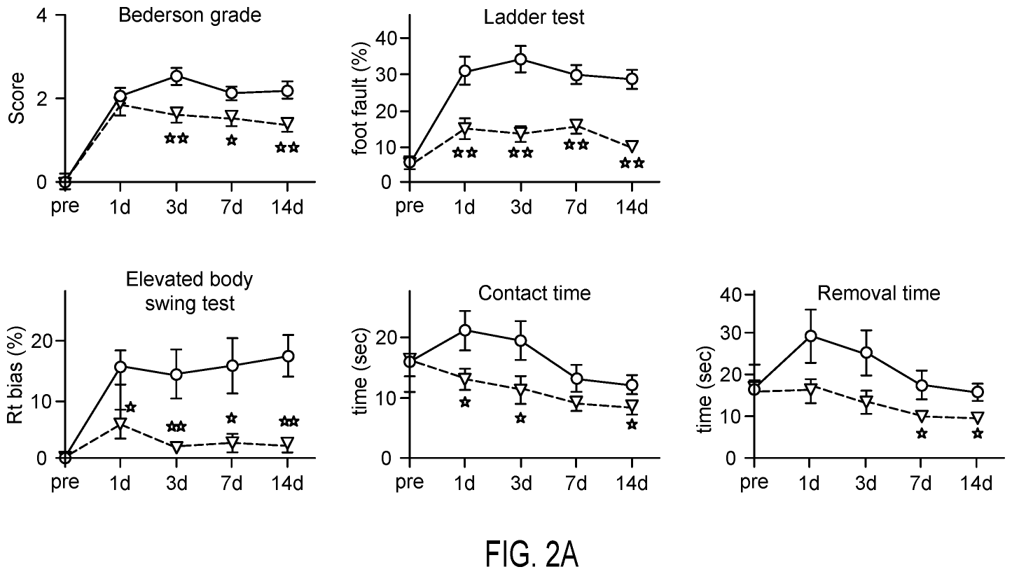

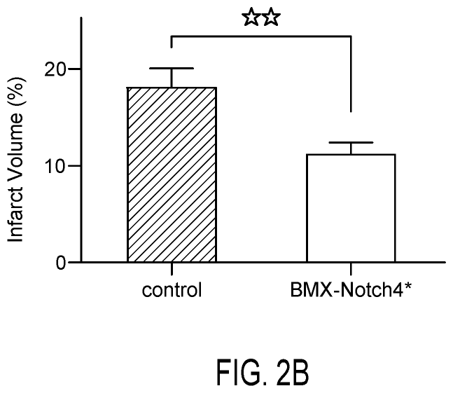

Similar results were observed in arterial activation of Notch4 signaling. Notch4* expression promoted neurological recovery, and reduced infarct volume in dMCAO model mice. Arterial activation of Notch4 improved neurological recovery after dMCAO. Neurological functions were assessed (FIG. 2A), including by modified bederson's grading, elevated body swing test, ladder test, and adhesive test. H&E staining showed less infarct lesions in BMX-Notch4* mice, compared with control mice. Quantification of infarct volume showed significantly less (p<0.01) infarct volume in BMX-Notch4* mice, compared with control mice (FIG. 2B).

Vascular casting of arteries in showed more prominent remodeling of collaterals in BMX-Notch4* mice, compared to that of control mice. Enlarged images clearly showed that collaterals in BMX-Notch4* mice are more prominent and tortuous, compared to that of control mice.

These results demonstrate that the induction of Notch signaling, prior to and following acute ischemic stroke, dramatically improves recovery of the injured mice, with greater restoration of blood flow and reduced infarc volume. This data suggests a preventative effect as well as a therapeutic effect.

### Example 3. Post-Ischemic Notch Signaling Activation Promotes Neurological Recovery, Reduction of Infarct Volume, and Increased Growth of Collateral Arteries

The previous results demonstrate that improved outcomes following ischemic injury in the brain may be achieved by the activation of Notch signaling, prior to and following injury. To demonstrate that this effect can be applied in a post-injury treatment context, an experiment was performed wherein Notch activation was induced after ischemic insult. In these trials, control and mutant Notch mice were subjected to dMCAO, followed by induction of Notch ICD expression by tamoxifen administration. To establish Notch signaling activation after stroke onset, tamoxifen (2 mg/body, ip) was injected on two consecutive days immediately after dMCAO surgery.

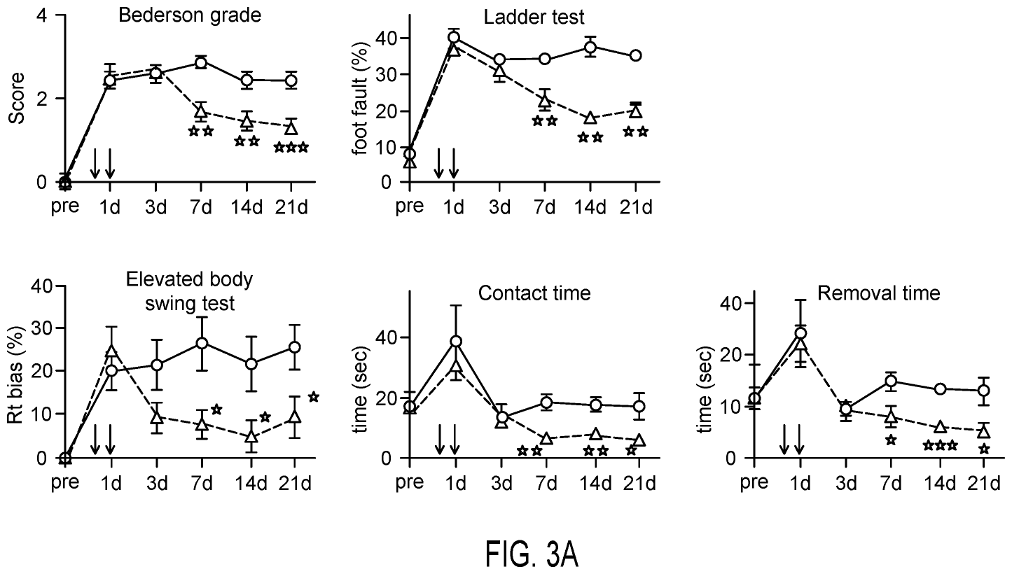

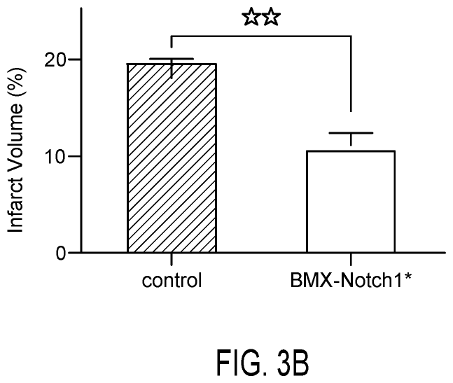

Arterial activation of Notch1 after ischemic injury improved neurological recovery. Prior to and following dMCAO ischemic injury, neurological functions were assessed (FIG. 3A), including by modified bederson's grading, elevated body swing test, ladder test, and adhesive test. BMX-Notch1 ICD mice showed significantly better neurological recovery, compared to that of control mice. Since Notch1 signaling activation was attained gradually after stroke, the difference between two groups became significant from 7 days after stroke. H&E staining showed less infarct lesions in BMX-Notch1 ICD mice in which Notch1 signaling upregulation was induced after stroke, compared with that of control mice. Quantification of infarct volume determined by H&E staining showed significantly less infarct volume in BMX-Notch1 ICD mice, compared with control mice (FIG. 3B).

Imaging of vascular casting of arteries showed more prominent remodeling of collaterals in the BMX-Notch1 ICD mice, when Notch1 ICD was expressed after experimental stroke, compared to that of control mice. Images clearly showed that collaterals in BMX-Notch1 ICD mice are more prominent and tortuous, compared to that of control mice.

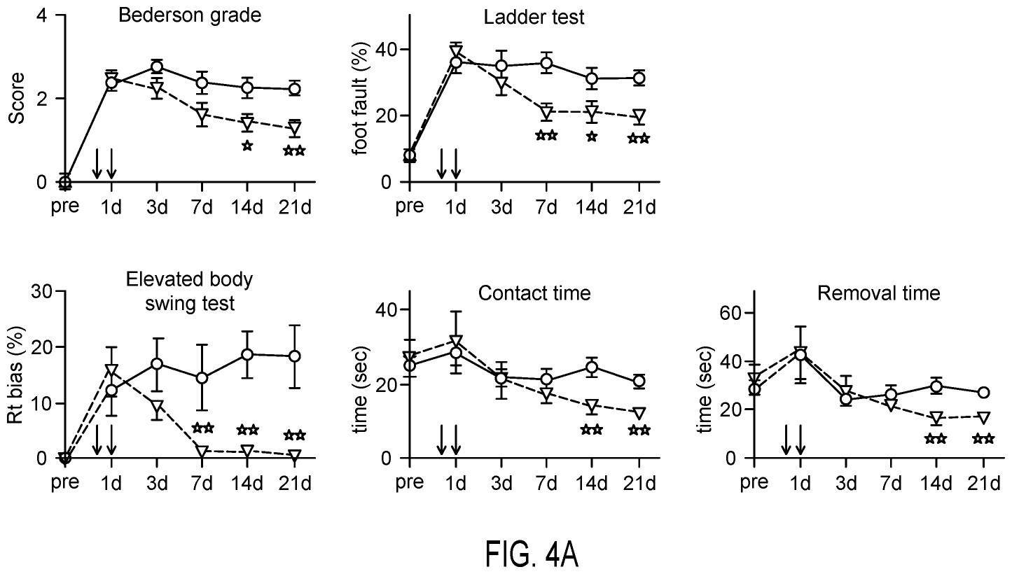

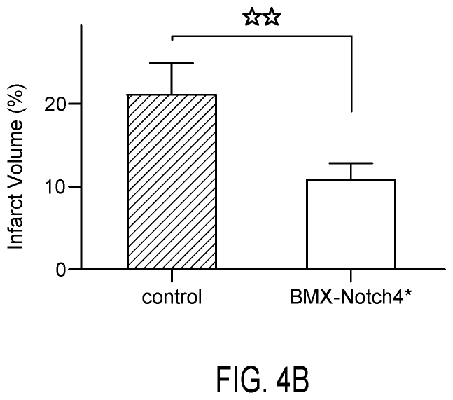

Similarly, post-ischemic Notch4 signaling activation initiated after ischemic insult promoted neurological recovery, reduction of infarct volume, and improved growth of collateral arteries. Neurological functions were assessed (FIG. 4A), including by modified bederson's grading, elevated body swing test, ladder test, and adhesive test before and after MCAO. Arterial activation of Notch4 was induced after ischemic injury by tamoxifen injection. Significantly improved neurological recovery was observed in the BMX-Notch4* mice. Since Notch4 signaling activation was attained gradually after stroke, the difference between two groups become significant from 7 days after stroke. HE staining showed less infarct lesions in BMX-Notch4* mice which notch4 signaling upregulation was attained after stroke, compared with that of control mice. Quantification of infarct volume determined by H&E staining showed significantly less infarct volume in BMX-Notch4* mice (FIG. 4B).

### Example 4. Durable Effects of Transient Notch Signaling Induction on Post-Ischemic Recovery

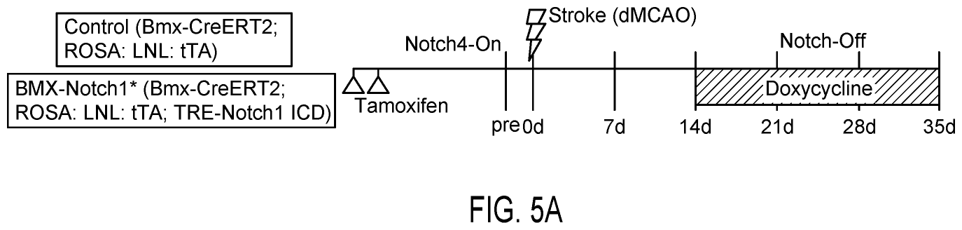

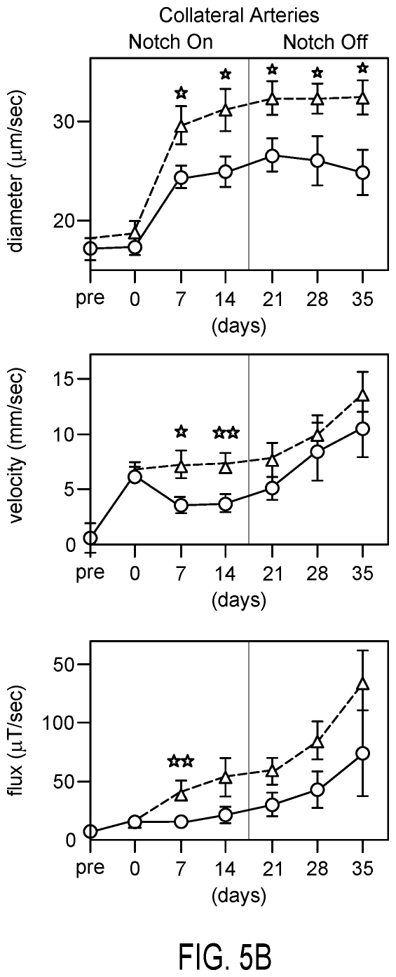

To further investigate the effects of upregulated Notch signaling following ischemic injury in the brain, an experiment was performed as summarized in FIG. 5A. Fourteen days prior to dMCAO, Notch signaling was induced in BMX-Notch1 mice by tamoxifen administration. Fourteen days following dMCAO, Notch1 ICD expression was turned off by means of the Tet-OFF expression construct and administration of doxycycline.

Arterial expression of Notch1 ICD promoted growth of collaterals, and preserved cerebral blood flow, despite turning off Notch1 ICD.

Collateral artery diameter, velocity, and flux (FIG. 5B) were all improved in BMX-Notch1 ICD mice, compared to control mice. Significant improvements in vascular size and function persisted after Notch1 ICD was turned off, demonstrating a lasting therapeutic effect from Notch activation in the period following injury.

Similarly, vascular casting of arteries showed more prominent remodeling of collaterals in BMX-Notch4* mice, compared to that of control mice, were sustained despite turning off Notch4* expression 14 days after dMCAO. Enlarged images clearly showed that the more prominent and tortuous collaterals in BMX-Notch4* mice, compared to that of control mice, were sustained long after Notch4* expression was turned off.

### Example 5. Therapeutic Effects of Notch Signaling Activation by Administration of a Notch Ligand

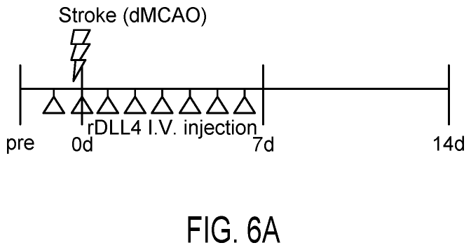

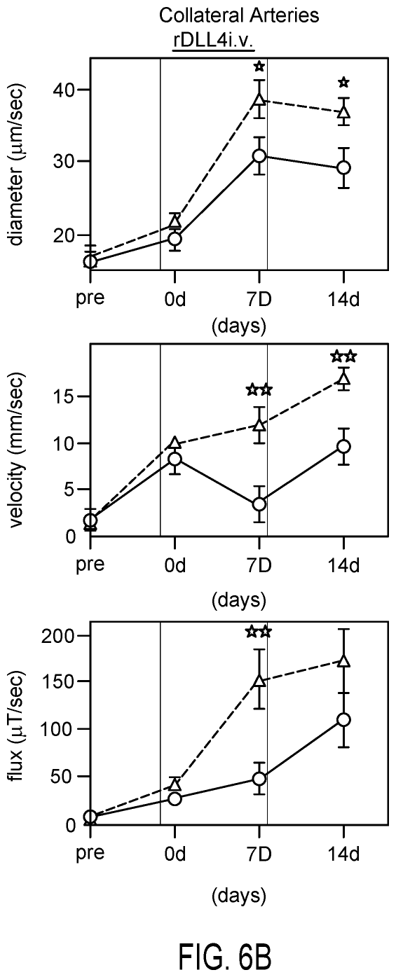

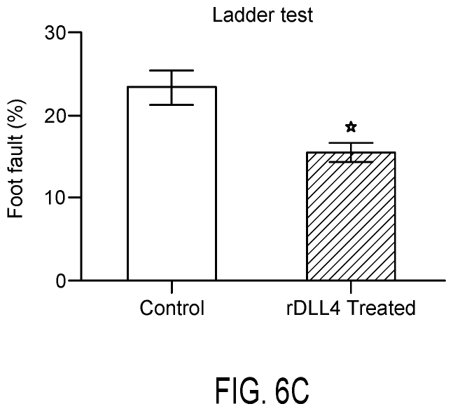

The foregoing experiments demonstrated the therapeutic effects of Notch signaling in ischemic brain injury by genetic approaches. To further demonstrate the therapeutic effects of upregulated Notch signaling following ischemic brain injury, recombinant mouse DLL4 was administered to wild type mice (C57BL/6Jstrain) daily, starting from the day before dMCAO for eight days, by intravenous injection at a dosage of 1 microgram per gram body weight for the first three doses and 0.8 micrograms per day thereafter, as summarized in FIG. 6A. Collateral artery size and function was assessed at seven and fourteen days following injury. In mice treated with rDLL4, collateral artery diameter, velocity, and flux (FIG. 6B) was significantly improved at day 7, at which time rDLL4 administration was halted. Significant improvements in the treated mice persisted at day 14. Neurological function, as assessed by the ladder test at day 15 and 17, was higher in the treated mice (FIG. 6C).

These results demonstrate that exogenous application of Notch signaling activators can have substantial and durable therapeutic effects in treating ischemic injury.

### Example 6. Upregulation of Notch Signaling Improves Recovery in Acute Limb Ischemia Model

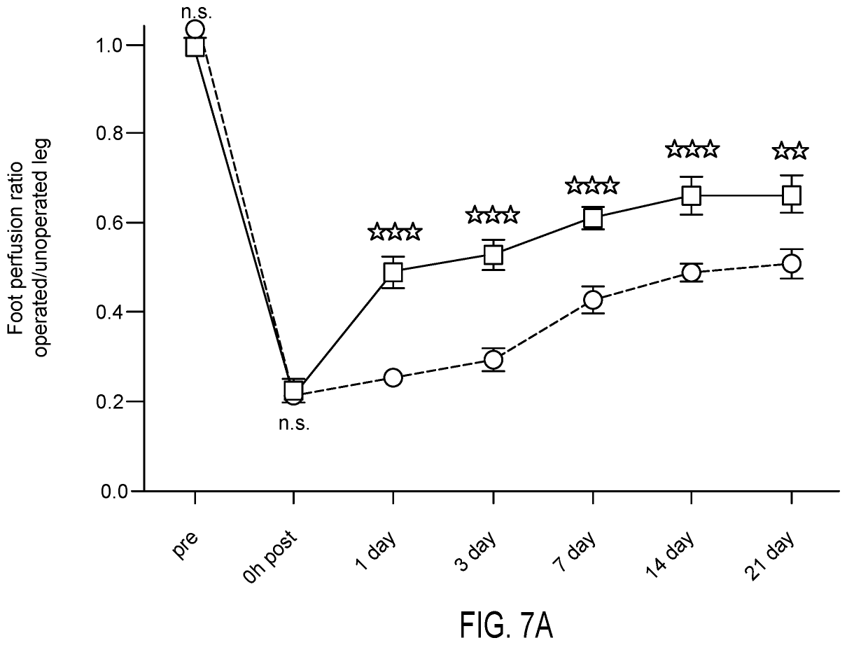

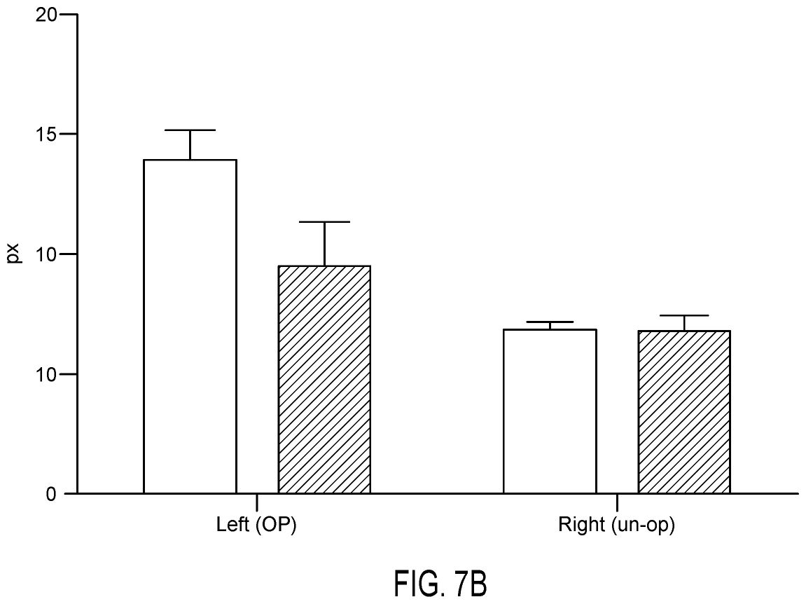

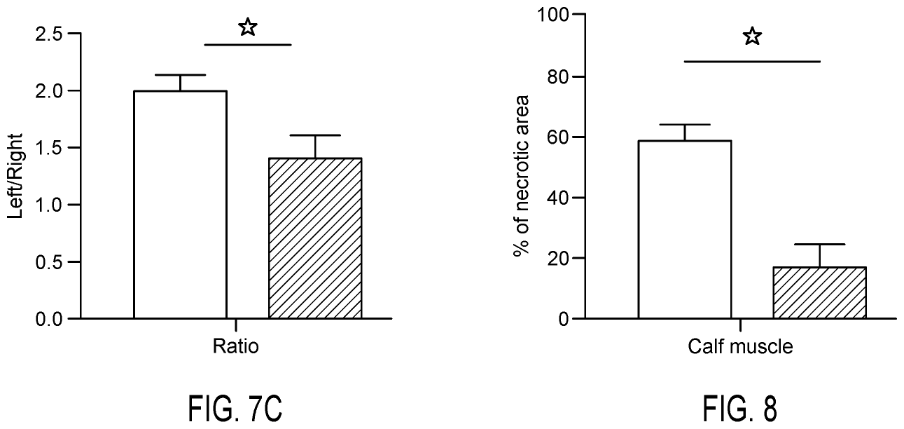

To study the therapeutic potential of arterial Notch signaling in acute limb ischemia, BMX-Notch4* mice and control mice were subjected to experimental femoral artery occlusion (EFAO) in the left hindlimb. Notch4* expression in the BMX-Notch4* mice were induced by tamoxifen administration 14 days prior to FFAO. Following EFAO, foot blood flow and hindlimb artery diameter were measured in the operated and unoperated limbs. FIG. 7A depicts improved blood flow in the ischemic foot of Notch4* expressing mice. FIG. 7B depicts improved recovery in hindlimb artery diameter in BMX-Notch4* mice, while no off-target effect was seen in the unoperated limb. FIG. 7C depicts the ratio in the change of collateral artery diameter after EFAO between operated and unoperated limbs. FIG. 8 depicts significantly reduced muscle necrosis in BMX-Notch4* mice at day-7 after EFAO.

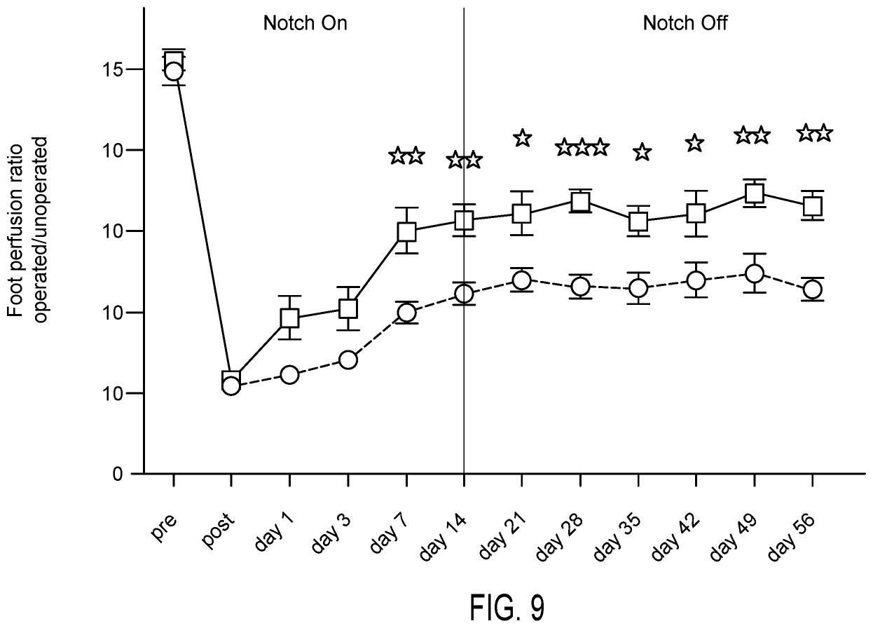

In a related experiment, the persistent effects of Notch4 activation were investigated in an acute limb ischemia model by subjecting BMX-Notch4* mice and control mice to EFAO in one limb. BMX Notch4* mice were injected with tamoxifen 13 and 14 days prior to EFAO procedure to induce Notch4* expression. Fourteen days after EFAO, Notch4* expression was turned off by administration of doxycycline (in chow). Notch4* expression was likely reduced over a period of a few days following doxycycline administration. Prior to, and following surgical injury, foot perfusion was measured in the operated and unoperated limbs by laser doppler perfusion imaging, or LDPI, as known in the art. The ratio of foot perfusion in the operated to unoperated limb provides a measure of foot blood flow recovery. Compared to control mice, foot blood flow was greater in the ischemic limbs of the BMX-Notch4* mice, becoming significantly improved by day 7 and remaining so for the remainder of the 56 day time course of measurement (FIG. 9). Despite turning off Notch 4* expression 14 days after surgery, the significant improvements in blood flow persisted through the period of measurement, well after Notch4* was turned off.

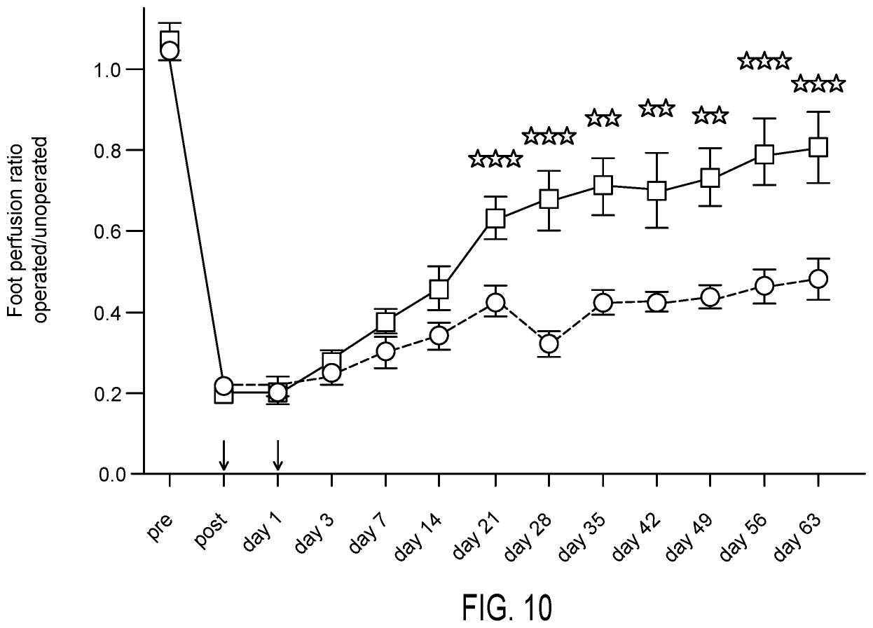

In a separate experiment, the therapeutic effects of Notch activation were investigated in an acute limb ischemia model by subjecting BMX-Notch4* mice and control mice to EFAO in one limb. Immediately following the EFAO procedure, tamoxifen administration was performed to induce Notch4* expression in the BMX-Notch4* mice. Prior to, and following surgical injury, foot perfusion was measured in the operated and unoperated limbs by (laser doppler perfusion imaging, or LDPI, as known in the art. The ratio of foot perfusion in the operated to unoperated limb provides a measure of foot blood flow recovery. As depicted in FIG. 10, significant improvements in foot perfusion following EFAO were observed in the Notch4*-expressing mice by day 21. These improvements persisted through the period of measurement, day 63.

These results demonstrate that the preventive and therapeutic effects of Notch signaling activation in the brain following ischemic injury are also applicable in other ischemic sites, and the results confirm that the effects can work preventatively, therapeutically, and also are durable, persisting well after Notch4 activation has ceased.

### Exemplary Embodiments

In one embodiment, the scope of the invention encompasses a Notch-activating agent for use in the treatment of a vascular disease, wherein the vascular disease is an ischemic condition, wherein the ischemic condition may be cardiac ischemia, ischemia of the brain, ischemia of the limb, or carotid artery disease.

In one embodiment, the scope of the invention encompasses a Notch-activating agent for use in the treatment of a vascular disease, wherein the vascular disease is an ischemic condition of the brain, wherein the ischemic condition may be acute ischemic stroke, transient ischemic attack, micro-infarct, vascular dementia, or cerebrovascular disease.

In one embodiment, the scope of the invention encompasses a Notch-activating agent for use in the treatment of a vascular disease, wherein the vascular disease is selected from the group consisting of peripheral artery disease, renal dysfunction or renal disease encompassing diminished blood flow; a vascular condition of the eye; diminished blood flow in the spleen; mesenteric artery occlusion; intestinal infarction; small vessel disease; and reduced blood flow in a vessel as a result of atherosclerosis or diabetes.

In one embodiment, the scope of the invention encompasses a Notch-activating agent for use in the treatment of a vascular disease, wherein the Notch-activating agent is a Notch ligand. In one embodiment, the Notch ligand is selected from the group consisting of delta-like ligand 1, delta-like ligand 2, delta-like 3, delta-like ligand 4, Jagged-1, Jagged-2, and a Notch-activating variant of the foregoing.

In one embodiment, the scope of the invention encompasses a Notch-activating agent for use in the treatment of a vascular disease, wherein the Notch-activating agent is a Notch intracellular domain. In one embodiment, the Notch intracellular domain is selected from the group consisting of a Notch 1, Notch 2, Notch 3, and Notch 4 intracellular domain, and a Notch-activating variant of the foregoing.

In one embodiment, the scope of the invention encompasses a Notch-activating agent for use in the treatment of a vascular disease, wherein the Notch-activating agent is selected from the group consisting of N-methylhemeanthidine chloride, valproic acid, resveratrol, triacetyl resveratrol, phenethyl isothiocyanate, and Yhhu-3792.

In one embodiment, the scope of the invention encompasses a Notch-activating agent for use in the treatment of a vascular disease, wherein the vascular disease is an ischemic condition, wherein the ischemic condition is selected from the group consisting of cardiac ischemia, ischemia of the brain, ischemia of the limb, or carotid artery disease, wherein the ischemic condition of the brain is selected from the group consisting of acute ischemic stroke, transient ischemic attack, micro-infarct, vascular dementia, or cerebrovascular disease; wherein the Notch-activating agent is a Notch ligand; wherein the Notch ligand is selected from the group consisting of delta-like ligand 1, delta-like ligand 2, delta-like 3, delta-like ligand 4, Jagged-1, Jagged-2, and a Notch-activating variant of the foregoing.

In one embodiment, the scope of the invention encompasses a drug delivery device, the drug delivery device being functionalized with one or more Notch-activating agents or coated with composition that elutes one or more Notch-activating agents when exposed to physiological conditions; wherein the Notch-activating agent is selected from the group consisting of a Notch ligand, a Notch intracellular domain, a small molecule activator of Notch, and an antibody, wherein, the Notch ligand is selected from the group consisting of delta-like ligand 1, delta-like ligand 2, delta-like 3, delta-like ligand 4, Jagged-1, Jagged-2, and a Notch-activating variants of the foregoing; wherein the device may be a drug-eluting balloon or a stent.

In one embodiment, the scope of the invention encompasses an a red blood cell functionalized with one or more Notch-activating agents. In one embodiment, the Notch-activating agent is a Notch ligand, In one embodiment, the Notch ligand is selected from the group consisting of delta-like ligand 1, delta-like ligand 2, delta-like 3, delta-like ligand 4, Jagged-1, Jagged-2, and a Notch-activating variant of the foregoing.

All patents, patent applications, and publications cited in this specification are herein incorporated by reference to the same extent as if each independent patent application, or publication was specifically and individually indicated to be incorporated by reference. The disclosed embodiments are presented for purposes of illustration and not limitation. While the invention has been described with reference to the described embodiments thereof, it will be appreciated by those of skill in the art that modifications can be made to the structure and elements of the invention without departing from the spirit and scope of the invention as a whole.

1-49. (canceled)

50. A method of increasing blood flow in a tissue of a subject,
wherein the tissue is afflicted with a condition wherein blood flow is reduced, impeded, or blocked in one or more blood vessels of the tissue;
comprising administering a pharmaceutically effective amount of a Notch-activating agent to arterial cells in the tissue.

51. The method of claim 50, wherein
the increased blood flow is achieved by any of vessel dilation; arteriogenesis; collateral arterial growth; remodeling of collateral vessels into conduit arteries; enhancing the conductance of blood vessels; or reducing the resistance of blood vessels.

52. The method of claim 50, wherein
the vascular condition is an ischemic condition.

53. The method of claim 52, wherein
the ischemic condition is cardiac ischemia.

54. The method of claim 53, wherein
the cardiac ischemia comprises stable angina, unstable angina, acute coronary syndrome, angina pectoris, myocardial infarction, and ischemia resulting from atherosclerosis.

55. The method of claim 51, wherein
the ischemic condition is ischemia of the brain.

56. The method claim 55, wherein
the ischemia of the brain is acute ischemic stroke, transient ischemic attack, micro-infarct, vascular dementia, or cerebrovascular disease.

57. The method of claim 51, wherein
the ischemic condition is ischemia of the limb.

58. The method of claim 51, wherein
the ischemic condition is carotid artery disease.

59. The method of claim 50, wherein
the condition is selected from the group consisting of peripheral artery disease, renal dysfunction or renal disease encompassing diminished blood flow; a vascular condition of the eye; diminished blood flow in the spleen; mesenteric artery occlusion; intestinal infarction; small vessel disease; and reduced blood flow in a vessel as a result of atherosclerosis or diabetes.

60. The method of claim 50, wherein
the Notch-activating agent is an activator of Notch 1.

61. The method of claim 50, wherein
the Notch-activating agent is an activator of Notch 4.

62. The method of claim 50, wherein
the Notch-activating agent is a Notch ligand.

63. The method of claim 62, wherein
the Notch-activating agent is selected from the group consisting of delta-like ligand 1, delta-like ligand 2, delta-like 3, delta-like ligand 4, Jagged-1, Jagged-2, and a Notch-activating variant of the foregoing.

64. The method of claim 50
the Notch-activating agent is a Notch intracellular domain.

65. The method of claim 64, wherein
the Notch-activating agent is selected from the group consisting of a Notch 1 intracellular domain, a Notch 2 intracellular domain, a Notch 3 intracellular domain, and a Notch 4 intracellular domain.

66. The method of claim 50, wherein
the Notch-activating agent is selected from the group consisting of N-methylhemeanthidine chloride, valproic acid, resveratrol, triacetyl resveratrol, phenethyl isothiocyanate, and Yhhu-3792.

67. The method of claim 50, wherein
the Notch activating agent comprises a nucleic acid construct that codes for a Notch-activating protein, or a downstream effector of Notch signaling.

68. The method of claim 50, wherein
the treatment is a preventative treatment.

69. The method of claim 50, wherein
the treatment is administered intravenously.

70. The method of claim 50, wherein
the Notch-activating agent is administered locally to a blood vessel in the tissue.

71. The method of claim 70, wherein
the Notch-activating agent is administered by a drug eluting balloon.

72. The method of claim 70, wherein
the Notch-activating agent is administered on a stent.

73. The method of claim 70, wherein
the Notch-activating agent is delivered in a film wrapped around a blood vessel in the target tissue.

74. The method of claim 50, wherein
the Notch-activating agent is a red blood cell, platelet, or synthetic mimic thereof that is functionalized with a Notch-activating agent.

75. The method of claim 50, wherein
the Notch-activating agent is conjugated to an antibody or antigen-binding fragment thereof, wherein the antibody or antigen binding fragment selectively binds to a ligand present in arterial cells.

76. The method of claim 74, wherein
the ligand present in arterial cells comprises CD34, Adhesion Molecule 1, or cross linked fibrin.
